(19) United States
(12) Patent Application Publication
(10) Pub. No.: US 2016/0011166 A1
(43) Pub. Date: Jan. 14, 2016

(54) METHOD FOR IDENTIFYING THE OAK SPECIES OF AN OAK WOOD SAMPLE

(71) Applicants: SEGUIN MOREAU & Cº, Merpins (FR); INSTITUT POLYTECHNIQUE DE BORDEAUX, Talence (FR); E. REMY MARTIN & C°, Cognac (FR); UNIVERSITE DE BORDEAUX, Bordeaux (FR)

(72) Inventors: Axel MARCHAL, Bordeaux (FR); Denis DUBOURDIEU, Beguey (FR)

(21) Appl. No.: 14/795,609

(22) Filed: Jul. 9, 2015

(30) Foreign Application Priority Data
Jul. 9, 2014 (FR) 14/56606

(51) Int. Cl. G01N 33/46; G01N 30/72; G01N 33/00

(52) U.S. Cl. CPC G01N 33/46; G01N 33/0098; G01N 30/7206; G01N 30/7233; G01N 2030/025

(57) ABSTRACT

The invention concerns a method for identifying the oak species including the following steps of: Providing an oak wood sample; Preparing said sample; Performing at least one analysis on the wood sample so as: to identify: from one to three molecules belonging to a 1^{st} category of molecules defined by the derivatives of oleanane-type triterpenes which derive from an aglycone of empirical formula C₃₀H₄₈O₆, from one to three molecules belonging to a 2^{nd} category of molecules defined by the derivatives of oleanane-type triterpenes which derive from an aglycone of empirical formula C₃₀H₄₆O₇, as well as derivatives of oleanane-type triterpenes which derive from an aglycone of empirical formula C₃₀H₄₆O₈, then to determine the concentration of the identified molecules; Determining a ratio R; Identifying the oak species.

The present invention concerns a method for identifying the oak species of an oak wood sample.

Oaks are hardwood trees. The oak wood has a mass density comprised between 0.75 and 0.85 g/cm³. This has the advantage of providing a material that is very resistant and hard for various applications such as, for example, cabinet making, joinery, parquetry, or still cooperage. Furthermore, the oak is particularly appreciated for being resistant to insects and fungi thanks to its high content of tannins.

Among the oak species, the sessile oak (Quercus petraea), also known under the name of <<durmast oak>>, and the pedunculate oak (Quercus robur) are the two most wide spread species that are encountered in the European forests.

The pedunculate and sessile oak woods have been used in shipbuilding until the 19^{st} century and have represented the main species of wood used in the construction of frameworks in Europe.

Nowadays, these oak woods are commonly used in joinery, parquetry, veneer production, as well as in oenology.

More specifically, as regards oenology, there are two major applications of the oak wood.

First of all, wines and spirit beverages (such as cognac, whisky, spirit) are aged in containers made of oak wood called for example, according to their shape and their volume: barrels, kegs and casks. Most of these containers are made of sessile or pedunculate oak wood. Indeed, aging is beneficial to the quality of wines and spirit beverages, thanks to the extraction of volatile and non-volatile compounds from the wood and to the complex transformations that take place during the aging in-wood.

Another oenological application of the oak wood consists in introducing pieces of this wood in wines and spirit beverages in order to enrich them with the chemical molecules present in said wood and thus modify their organoleptic qualities. In this technical field, these products are generally designated under the name <<alternative products>>) and are used in the form of chips, shavings, powders, as well as more voluminous pieces such as sticks.

Hence, both sessile and pedunculate oak species are used for the manufacture of containers for oenology and also for the production of the aforementioned alternative products.

Because of their respective characteristics, the sessile oak is generally reserved for making barrels for wine whereas the pedunculate oak is reserved for making barrels for spirit beverages.

Although these two sessile and pedunculate oak species present distinct morphological characteristics, for example at the trunk, the branches, the bark or even their foliage, it is difficult to unambiguously discriminate them from one another through visual examination.

Furthermore, in most forests (for example in French forests), these two oak species are often mixed. This is why it is not possible to rely on the forest from which the oak wood comes when determining its species. Indeed, genetic analyses have shown that, within some forests known to be populated by sessile oaks, a significant quantity of pedunculate oaks also exists.

This is why the only method for unambiguously identifying the oak species of an oak wood sample lies on these genetic analyses. They require to be carried out on fresh wood or, ideally, on leaves, which in practical terms, is not always easy to implement. Furthermore, these genetic analyses do not provide an immediate result, but on the contrary, require some time. Finally, these analyses are expensive.

However, considering the very different uses because of the diversity of the beverages which are stored in containers made of oak wood, it is essential for the cooper to know precisely and also to guarantee the oak species that is used for the manufacture of his containers.

Indeed, regarding cooperage, the characteristics and the quality of the raw material (that is to say the oak wood) that is used for the storage of alcoholic beverages, condition significantly the quality of the final product, namely the alcoholic beverage.

It is easily understood that it is essential for the cooper to know accurately the species of the oak wood that is available for the manufacture of his wooden containers.

To date, the only method for identifying an oak species with certainty consists in carrying out genetic analyses which are, as has been mentioned above, time-consuming and tedious.

The present invention intends to overcome the drawbacks of genetic analyses which have been detailed above by providing a new method for identifying the oak species of an oak wood sample.

The identification method according to the invention has the advantages of being easy to implement and of obtaining the result very quickly.

In addition, the method according to the invention guarantees a perfectly reliable result and this, in particular, in contrast with a visual examination of a sample of an oak element such as for example its leaves or its bark.

More precisely, the method for identifying the oak species of an oak wood sample is characterized in that it comprises at least the following steps of:

a) Providing an oak wood sample;

b) Preparing the oak wood sample in a suitable manner for an analysis for determining the concentration of derivatives of oleanane-type triterpenes which are present in said oak wood sample;

c) Performing at least one analysis on the oak wood sample that has been prepared at step b) so as:
to identify:

i. from one to three molecules belonging to a 1^{st} category of molecules, said one to three molecules being among the three most abundant molecules of all the molecules of said 1^{st} category of molecules present in said oak wood sample, said 1^{st} category of molecules being defined by all derivatives of oleanane-type triterpenes which derive from an aglycone of empirical formula C₃₀H₄₈O₆,

ii. from one to three molecules belonging to a 2^{nd} category of molecules, said one to three molecules being among the three most abundant molecules of all the molecules of said 2^{nd} category of molecules present in said oak wood sample, said 2^{nd} category of molecules being defined by all derivatives of oleanane-type triterpenes which derive from an aglycone of empirical formula C₃₀H₄₆O₇, as well as derivatives of oleanane-type triterpenes which derive from an aglycone of empirical formula C₃₀H₄₆O₈, then
to determine the concentration:
of each of said one to three molecules of the 1^{st} category of molecules which have been identified;
of each of said one to three molecules of the 2^{nd} category of molecules which have been identified;

d) Determining a ratio R, said ratio R corresponding to the ratio of the concentration or the sum of the concentrations of at least one of said one to three molecules of the 1^{st} category of molecules which have been identified, to the concentration or the sum of the concentrations of at least one of said one to three molecules of the 2^{nd} category of molecules which have been identified;

e) Identifying the species of the oak wood sample according to the value of the ratio R that has been determined at step d) in the following manner:
If the ratio R is lower than 0.316, then the oak wood sample belongs to the pedunculate species,
If the ratio R is higher than 3.162, then the oak wood sample belongs to the sessile species.

In the context of the present invention, it is recalled that triterpenes are hydrocarbons with thirty atoms of carbon resulting from the condensation of six isoprene molecules.

One of the advantages of the method for identifying the oak species according to the invention lies in the fact that, for almost all the oak wood samples which will be analyzed, a result (namely, the determination of whether the sample is a sessile oak or a pedunculate oak) will be obtained without any ambiguity.

Preferably, the 1^{st} category of molecules is defined by all derivatives of oleanane-type triterpenes which derive from the arjungenin, the sericic acid, as well as the isomers of the arjungenin and those of the sericic acid.

Preferably, the 2^{nd} category of molecules is defined by all derivatives of oleanane-type triterpenes which derive from the bartogenic acid, the isomers of the bartogenic acid, the trachelosperogenin D and the isomers of the trachelosperogenin D.

The formulae of the arjungenin, the sericic acid, the bartogenic acid and the trachelosperogenin D are detailed below.

The formula (A) below corresponds to the arjungenin.

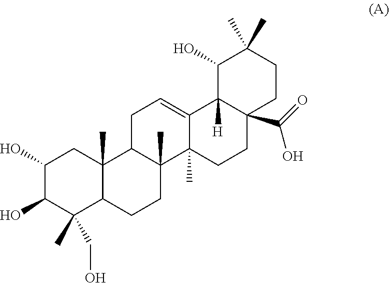

The formula (B) below corresponds to the sericic acid.

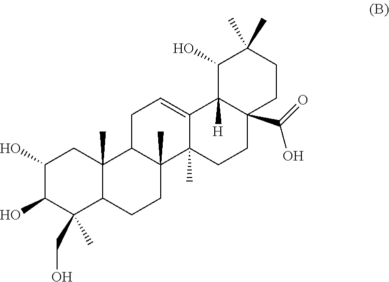

The formula (C) below corresponds to the bartogenic acid.

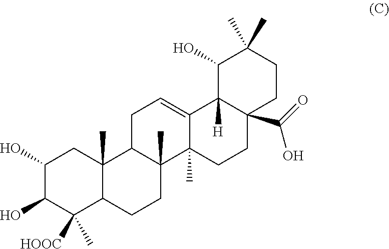

The formula (D) below corresponds to the trachelosperogenin D.

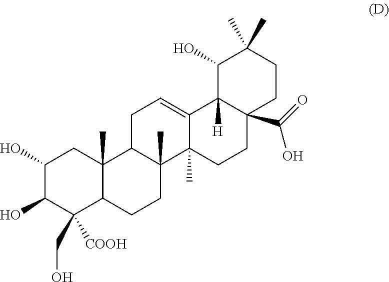

In one embodiment of the invention, the 1^{st} category of molecules is defined by all molecules:
of formula (I) as detailed below,

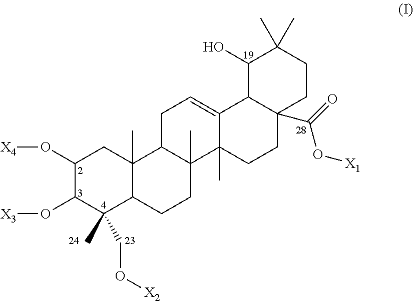

wherein:
X1 represents a glucose group or a hydrogen,
X2 represents a gallate group or a hydrogen,
X3 represents a hydrogen or a glucose group or a gallate group or a glucose-gallate group,
X4 represents a hydrogen or a gallate group,
of formula (II) as detailed below,

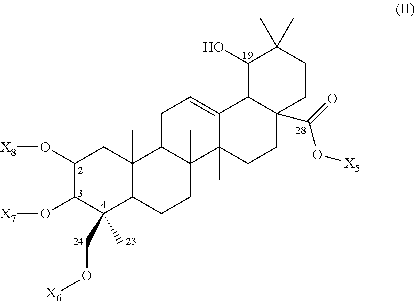

wherein:

X5 represents a glucose group or a hydrogen,
X6 represents a gallate group or a hydrogen,
X7 represents a hydrogen or a glucose group or a gallate group or a glucose-gallate group,
X8 represents a hydrogen or a gallate group.

In one embodiment of the invention, the 2^{nd} category of molecules is defined by all molecules:
of formula (III) as detailed below,

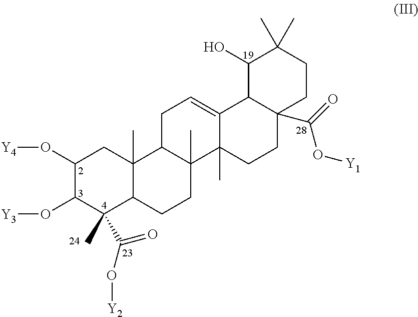

wherein:
Y1 represents a glucose group or a hydrogen,
Y2 represents a glucose group or a hydrogen,
Y3 represents a hydrogen or a gallate group or a glucose group or a glucose-gallate group,
Y4 represents a hydrogen or a gallate group,
of formula (IV) as detailed below,

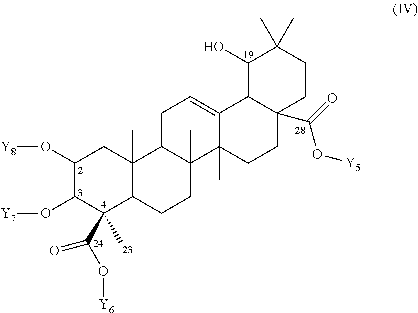

wherein:
Y5 represents a glucose group or a hydrogen,
Y6 represents a glucose group or a hydrogen,
Y7 represents a hydrogen or a glucose group or a gallate group or a glucose-gallate group,
Y8 represents a hydrogen or a gallate group,
of formula (V) as detailed below,

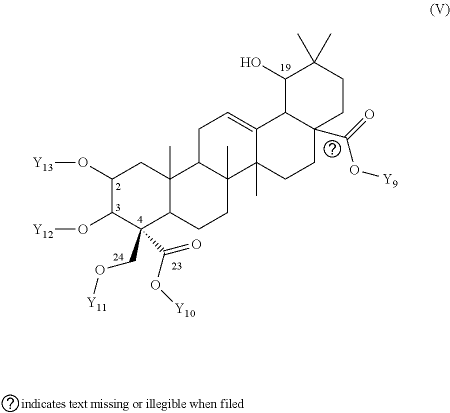

wherein:
Y9 represents a glucose group or a hydrogen,
Y10 represents a glucose group or a hydrogen,
Y11 represents a hydrogen or a gallate group,
Y12 represents a hydrogen or a glucose group or a gallate group or a glucose-gallate group,
Y13 represents a hydrogen or a gallate group,
of formula (VI) as detailed below,

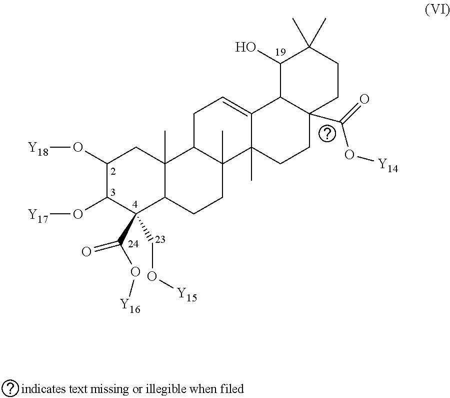

wherein:
Y14 represents a glucose group or a hydrogen,
Y15 represents a hydrogen or a gallate group,
Y16 represents a glucose group or a hydrogen,
Y17 represents a hydrogen or a glucose group or a gallate group or a glucose-gallate group,
Y18 represents a hydrogen or a gallate group.

In one embodiment of the invention, the 1^{st} category of molecules is defined by all the following molecules:
the quercotriterpenoside I (hereinafter abbreviated by QTT I) of formula (VII),
the quercotriterpenoside II (hereinafter abbreviated by QTT II) of formula (VIII), and
the quercotriterpenoside III (hereinafter abbreviated by QTT III) of formula (IX),

and wherein the formulae are detailed below.

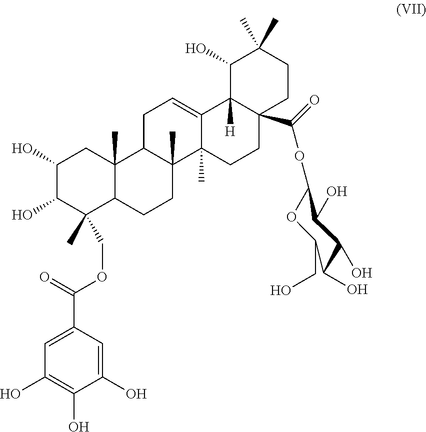

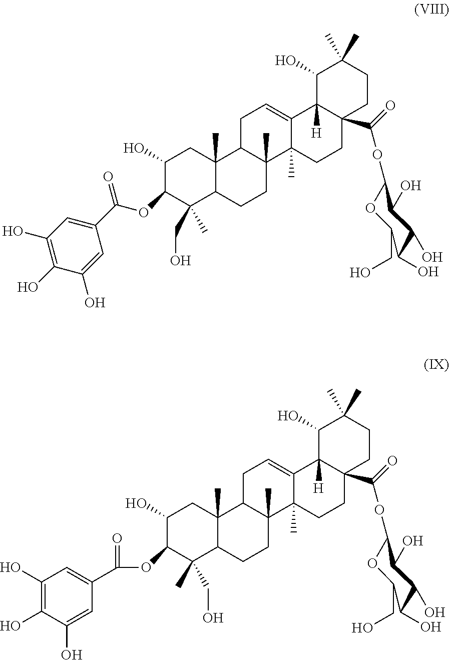

In one embodiment of the invention, the 2^{nd} category of molecules is defined only by the 28-glucosylated derivative of the bartogenic acid (hereinafter abbreviated by Glu-AB) of formula (X) as detailed below.

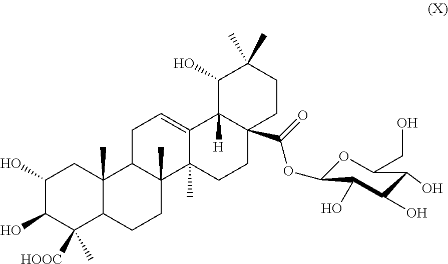

In one embodiment of the invention, the ratio R corresponds to the ratio of the concentration or the sum of the concentrations of at least one of the molecules chosen among the QTT I of formula (VII), the QTT II of formula (VIII) and the QTT III of formula (IX), to the concentration of the Glu-AB of formula (X).

In one embodiment of the invention, the ratio R corresponds to the ratio of the sum of the concentrations of the QTT I of formula (VII), the QTT II of formula (VIII) and the QTT III of formula (IX), to the concentration of the Glu-AB of formula (X).

The preparation of the oak wood sample is now further described.

The oak wood of the sample to be analyzed for the identification of the oak species may have quite various origins, in particular depending on the application to which the identification method according to the invention is intended. Indeed, it may for example consist of the two aforementioned oenological applications which are:
the identification of the oak species of alternative products;
the identification of the oak species of wooden containers in which wines and spirit beverages are aged.

Thus, in the context of the present invention, the oak wood of the sample may come from one single piece of wood such as a shook, or from a set of homogeneous pieces of wood such as for example a set of staves intended to the manufacture of a keg, or from a set of alternative products.

Of course, the examples that have been detailed above and which describe the origin of the wood constituting the sample do not limit the scope of the invention.

Indeed, the identification method according to the invention has the advantage of being able to be implemented over a whole oak wood sample, and this regardless of its nature and origin (for example, a piece of wood collected on an oak tree or coming from a product manufactured with wood such as a shook, a stave or other equivalent products).

Preferably, at step b) of the identification method according to the invention, the oak wood sample is prepared through a solid-liquid extraction.

It is possible to start by preparing the oak wood sample that is available by cutting it into chips, and then letting the thus obtained chips macerate in a liquid for a determined time duration. Advantageously, before the maceration, said chips are reduced to powder.

In one embodiment of the invention, the oak wood sample is left to dry, for a few hours (for example for at least 6 hours) at ambient temperature, for example at a temperature of at least 20° C.

The maceration of the oak wood may be performed in any suitable liquid, for example in water or still in a solvent such as an alcoholic solution. It may consist of a solution of ethanol, for example a solution of ethanol at 12% v/v. The solution may have an acidic pH, for example in the range of 3.5.

An example of a solution in which the oak wood sample is macerated, for example in form of chips or powder, consists of a solution of ethanol at 12% v/v further containing tartaric acid (for example at a concentration of 5 g/L) with a pH of 3.5. The pH of this solution is adjusted with soda, for example at a concentration of 5 mol/L. The concentration of the oak wood sample in this solution may be for example of 50 g/L.

The oak wood sample is macerated in a liquid for several hours, for example for a time duration of at least 48 hours, and this at the ambient temperature (namely about 20° C.).

Preferably, the maceration of the oak wood sample takes place in the darkness. This has the advantage of protecting some of the molecules of the oak wood which are sensitive to light and thereby avoiding their degradation before carrying out the analysis which is intended to determine their concentration; which might lead to an erroneous result of the oak species of the sample.

The example of embodiment for the maceration of the oak wood sample that has been described above does not limit the present invention. It consist of an embodiment that may be considered in the context of the present invention for preparing said sample in view of its analysis at step c) of the identification method which, recall again, consists in identifying and assaying (in other words extracting and quantifying) from one to three of the most abundant molecules among the molecules of the 1^{st} category of molecules and those of the 2^{nd} category which have been described above.

Indeed, the inventors of the present invention have observed that this embodiment for the maceration of the oak wood sample is suitable for carrying out the analysis of step c) since these maceration conditions are quite similar to the conditions of wine aging during which, recall again, volatile and non-volatile compounds of the wood are extracted.

Of course, other conditions for extracting the most abundant molecules of the 1^{st} category of molecules and those of the 2^{nd} category of molecules that are as effective as those described above may be implemented and are perfectly within the reach of those skilled in the art.

Thus, in the context of the present invention, the maceration of the wood sample may be carried out under other conditions of time duration and temperatures than those described above. For example, the maceration may be performed at temperatures that are higher than the ambient temperature (for example, comprised between about 50° C. and 80° C.), and this for a time duration that is shorter than the aforementioned one (for example for a few hours, namely between 5 and 10 hours).

At completion of the maceration, an oak wood macerate is obtained.

In one embodiment of the invention, in order to carry out the analysis of step c) of the identification method according to the invention, the oak wood macerate is diluted, for example through a 20-times dilution, advantageously in ultrapure water, and afterwards, it is filtered, for example through a PTFE (<<polytetrafluoroethylene>>) filter with a mesh diameter of 0.45 μm. This consists of a possible embodiment for finishing the preparation of the oak wood sample. Of course, other dilutions and other filtering means may be considered in the context of the present invention. These are perfectly known from those skilled in the art.

Of course, in the context of the present invention, it should be noted that the dilution of the oak wood macerate is optional. In other words, step b) of preparing the oak wood sample may be carried out without any dilution step.

For example, a dilution will not be necessary if the initial concentration of the crude extract of oak wood of the sample to be analyzed is lower than 100 mg/L after filtering. Thus, in this embodiment of the invention, the preparation step b) may consist of a maceration of the oak wood sample (for example in form of a powder) in a mixture of water and ethanol, the concentration of ethanol may be lower than the aforementioned concentration, and for example lower than 10% v/v.

Of course, the preparation of the oak wood sample is perfectly within the reach of those skilled in the art who will know exactly how to proceed for preparing said sample depending on the analyses that he will perform for determining the concentrations of the molecules of the 1^{st} and 2^{nd} categories of molecules which, recall again, have the common characteristic of being derivatives of oleanane-type triterpenes.

Thus, the embodiments for the preparation of the oak wood sample that have been described above correspond to examples of embodiments of step b) which may be considered in the context of the present invention but do not limit the scope of the identification method according to the invention.

The analysis of step c) of the identification method may consist of at least one chemical analysis which allows determining the concentration of the chemical molecules which are derivatives of oleanane-type triterpenes.

For example, it may consist of at least one analysis chosen among:
LC-MS analyses (namely a liquid chromatography analysis coupled to a mass spectrometry analysis),
GC-MS analyses (namely a gas chromatography analysis coupled to a mass spectrometry analysis),

NMR analyses (namely a nuclear magnetic resonance analysis).

Of course, carrying out these chemical analyses which are detailed above is perfectly within the reach of those skilled in the art who will know how to implement the adequate analysis protocol for identifying and then determining the concentration of the most abundant molecules of the 1^{st} category and 2^{nd} category of molecules as have been detailed above.

In other words, those skilled in the art know how to determine the concentration of derivatives of oleanane-type triterpenes present in a sample, and in particular in an oak wood sample, and this for example after having subjected said sample to a solid-liquid extraction.

In particular, as regards the liquid and/or gas chromatography analyses, those skilled in the art know how to realize the appropriate calibration curves, and when appropriate, the ratio of the surface areas of the chromatograms which are necessary to determine the concentrations of the most abundant molecules of the 1^{st} category of molecules and those of the 2^{nd} category of molecules he had identified in the oak wood sample to be analyzed.

Preferably, at step e) of the identification method, the decimal logarithm of the ratio R is calculated, and:
if the decimal logarithm of the thus calculated ratio R is higher than 0.5 then the oak wood sample belongs to the sessile species;
if the decimal logarithm of the thus calculated ratio R is lower than −0.5 then the oak wood sample belongs to the pedunculate species.

The decimal logarithm constitutes a means that allows to express the relative abundance of the identified molecules of the 1st and 2^{nd} categories of molecules.

The calculation of the decimal logarithm has the advantage of providing a result that is easy to read since the nature of the oak wood species is related to the sign of the thus obtained value: if the result is negative, then it will consist of a pedunculate oak, and if, on the contrary, the result is positive, then it will consist of a sessile oak.

It should be noted that the method for identifying the oak wood species has the following advantages:
the preparation of the oak wood sample may be carried out on fresh wood, but also on staves wood after drying and even after having been heated, and this in contrast with the genetic analyses which require fresh oak samples;
the preparation of the wood sample is simple and the analysis duration is very short, and this also in contrast with genetic analyses.

Thus, the use, in an industrial scale, of the method for identifying the oak species according to the invention allows a cooper to certify that his wooden containers (for example, kegs, tanks, barrels) are not made from a sessile oak or from a pedunculate oak, depending on whether they are intended, respectively, to aging of wines and spirits. Thus, adaption of the keg to its content is optimized.

The identification method according to the invention constitutes a solution that is particularly suitable for the technical field of cooperage, because it is simple to implement while remaining inexpensive and provides an identification result very quickly.

With the identification method according to the invention, it is possible to proceed without genetic analyses when it is desired to know without ambiguity the oak species to which an oak wood sample belongs.

Similarly, the identification method according to the invention is perfectly suitable for identifying the oak wood species in the context of another oenological application that has been mentioned above, namely the application that concerns the alternative products (chips, powders, sticks) introduced in wines and spirit beverages in order to modify their organoleptic qualities.

Thus, the identification method according to the invention allows for an easy in and unambiguous determination of the oak species constitutive of alternative products.

The present invention is by no way limited to the analysis of oak wood samples coming from these two oenological applications which have been mentioned above.

Thus, in the context of the present invention, it is perfectly possible to also consider implementing the method for identifying the oak species as described above for identifying the oak species in medical and/or pharmaceutical samples. Indeed, in various medical and/or pharmaceutical applications, it may be necessary to identify the oak wood species that some medical and/or pharmaceutical preparations may contain. In this case, those skilled in the art will also perfectly know how to prepare the oak wood sample coming from these medical and/or pharmaceutical preparations and then, identify and assay from one to three the most abundant molecules of the molecules of the 1^{st} and 2^{nd} categories of molecules as defined above.

### DESCRIPTION OF THE FIGURES

FIG. 1 is a diagram of the concentration of the QTT I of formula (VII) determined in each of the oak wood samples A to V.

FIG. 2 is a diagram of the concentration of the QTT II of formula (VIII) determined in each of the oak wood samples A to V.

FIG. 3 is a diagram of the concentration of the QTT III of formula (IX) for each of the oak wood samples A to V.

FIG. 4 is a diagram of the concentration of the Glu-AB of formula (X) for each of the oak wood samples A to V.

FIG. 5 is a diagram of the sum of the concentrations of QTT I, QTT II and QTT III.

FIG. 6 is a diagram of the decimal logarithm of the ratio R of each of the samples A to V.

An example of an implementation of the method for identifying the oak species of an oak wood sample according to the invention is detailed on the basis of the experimental part that follows.

### EXPERIMENTAL PART

Oak samples have been collected on 22 trees, in 6 French forests, by collecting, at the same time:
leaves, for determining the oak species by a genetic analysis, and
wood, for determining the oak species according to the identification method according to the invention.

Thus, 22 samples referenced from A to V, have been obtained.

Each of the 22 samples A to V has been subjected to:
a genetic analysis;
a method for identifying the oak species according to the invention.

In the context of this experimental part, the genetic analyses have served to confirm that the method for identifying the oak species according to the invention allows identifying, without error, the oak species of these different 22 oak wood samples. Indeed, the genetic analysis gives an unambiguous result of the species.

Thus, genetic analyses have been carried out on the oak wood samples A to V.

According to the genetic analyses:
the samples A to H are samples of the sessile oak species, and
the samples I to V are samples of the pedunculate oak species.

Moreover, the oak wood of each of the 22 samples A to V has been prepared in the following manner:
the collected oak wood has been reduced to powder;
afterwards, the oak wood powder has been added to an aqueous solution containing ethanol (12% v/v) at a concentration of 50 g/L, said aqueous solution further containing tartaric acid (at a concentration of 5 g/L) and had a pH of 3.5. The pH of this solution has been adjusted with soda (at a concentration of 5 mol/L);
the thus obtained solution has been left to macerate for 48 hours in the darkness;
afterwards, the thus obtained oak wood macerate has been diluted 20-times in ultrapure water and then filtered on a PTFE filter with a mesh diameter of 0.45 μm.

For each of the 22 oak wood samples that have been prepared, the concentration of the following molecules has been determined:
the QTT I, QTT II and QTT III, namely molecules belonging to the 1^{st} category of molecules of the identification method according to the invention, and
the Glu-AB, namely a molecule belonging to the 2^{nd} category of molecules of the identification method according to the invention.

The determination of these concentrations has been carried out by a liquid chromatography coupled with a Fourier transform mass spectrometry (LC-FTMS). After having established the calibration lines, the 4 molecules detailed above have been assayed simultaneously.

The analysis of each sample has lasted for about 7.5 minutes.

More precisely, the technical characteristics of the liquid chromatography have been as follows:
An ultra-high performance liquid chromatography platform comprising a HTC PAL type sample feeder (equipment from the company CTC Analytics AG) coupled with an Accela type pumping system.
A C18 type column has been used as a stationary phase, said column is commercialized by the company Thermo Fisher Scientific under the commercial name Hypersil Gold and has the following dimensions: 2.1 mm×100 mm and 1.9 μm for the size of the particles.
The mobile phase has consisted of a mixture of water and acetonitrile. The flow rate has been 600 μL/min and the volume percentage of acetonitrile in the mobile phase has varied in the following manner:

At start: 20% v/v;
30 seconds after the start of the analysis: 20% v/v;
4 minutes after the start of the analysis: 50% v/v;
4.1 minutes after the start of the analysis: 98% v/v;
6.1 minutes after the start of the analysis: 98% v/v;
6.2 minutes after the start of the analysis: 20% v/v;
7.5 minutes after the start of the analysis: 20% v/v.
The injection volume was 5 μL.
Each sample and each calibration level have been injected three times.

The ultra-high performance liquid chromatography device described above has been coupled with an Exactive type mass spectrometer, fitted with an Orbitrap analyzer and equipped with an electrospray ionization probe. These two equipments are commercialized by the company Thermo Fisher Scientific.

The mass spectrometry data have been acquired for 6 minutes in negative ionization FTMS mode (namely, <<Fourier Transform Mass Spectrometry>>).

The other parameters of the mass spectrometry analysis are detailed in Table 1 below.

TABLE 1
detailing the parameters of the mass spectrometry analysis

| FTMS parameter | Value | |
|---|---|---|
| Flow rate of the sheath gas | 75 | |
| Flow rate of the auxiliary gas | 18 | |
| Temperature of the ionization source | 320° | C. |
| Temperature of the capillary | 350° | C. |
| Ionization voltage | −3 | kV |
| Voltage of the capillary | −95 | V |
| Voltage of the tube lens | −190 | V |
| Voltage of the skimmer | −46 | V |
| Fragmentation energy by disassociation in the source | 20 | eV |
| Scanning range | 500-1200 | Th |

| | |
|---|---|
| Resolution | 25 000 |
| Value of the automatic gain control | 3.10⁶ |

In Table 1 above, the gases consist of dinitrogen and the gaseous flow rates are expressed in arbitrary units (that is to say, in the two first lines of Table 1).

In addition, as regards the fragmentation mode, an energy of 20 eV has been applied in the source in order to disassociate the possible adducts of the molecules to be quantified with the anions of the medium.

The resolution is defined as the m/Δm ratio at mid-height and is expressed above for an ion with a 200 Th m/z ratio.

An external calibration of the spectrometer using a <<Pierce® ESI Negative Ion Calibration>> (Thermo Fisher Scientific) calibration solution, has been performed before each series of analyses. Data have been processed using the Qualbrowser and Quanbrowser applications of the Xcalibur version 2.1 (Thermo Fisher Scientific) software.

The detection of the 4 aforementioned molecules has been carried out based on their exact theoretical mass, as well as on their respective theoretical retention time periods.

The surface areas of the peaks have been determined by automatic integration and the concentrations of the 4 aforementioned molecules have been determined while taking into account the dilution factor (20 times) detailed above.

Thus, the concentrations of the 4 aforementioned molecules present in the different oak wood samples A to V, which have been prepared as detailed above, have been automatically calculated.

Table 2 below details, for the samples A to V, the concentrations of QTT I, QTT II and QTT III, said concentrations being expressed in mg/L and in μg/g (μg/g means <<microgram/gram>>).

TABLE 2
detailing, for the samples A to V, the concentrations in mg/L
and in μg/g, of QTT I, QTT II and QTT III, as well as the
sum of the concentrations of QTT I, QTT II and QTT III

| | mg/L | μg/g | mg/L | μg/g | mg/L | μg/g | mg/L | μg/g |
|---|---|---|---|---|---|---|---|---|
| | QTT I | | QTT II | | QTT III | | TOTAL QTT | |
| A | 12.5 | 249.8 | 6.0 | 120.4 | 17.9 | 357.8 | 36.4 | 728.0 |
| B | 20.2 | 403.5 | 5.1 | 102.7 | 30.8 | 615.0 | 56.1 | 1121.2 |
| C | 41.6 | 832.5 | 70.9 | 1418.0 | 70.4 | 1408.7 | 183.0 | 3659.2 |
| D | 23.9 | 477.9 | 4.1 | 82.8 | 11.8 | 235.3 | 39.8 | 796.0 |
| E | 13.3 | 265.4 | 2.2 | 43.1 | 6.6 | 132.5 | 22.1 | 441.0 |
| F | 29.0 | 580.9 | 6.8 | 135.2 | 24.2 | 484.2 | 60.0 | 1200.2 |
| G | 40.7 | 814.0 | 66.0 | 1320.0 | 70.3 | 1405.2 | 177.0 | 3539.2 |
| H | 2.8 | 56.0 | 1.9 | 37.5 | 3.7 | 74.0 | 8.4 | 167.5 |
| I | 0.2 | 3.1 | 0.2 | 4.0 | 0.2 | 4.7 | 0.6 | 11.7 |
| J | 0.1 | 2.1 | 0.1 | 2.1 | 0.2 | 3.3 | 0.4 | 7.4 |
| K | 0.1 | 1.6 | 0.5 | 10.2 | 0.1 | 2.9 | 0.7 | 14.7 |
| L | 0.1 | 1.3 | 0.1 | 1.8 | 0.1 | 1.9 | 0.3 | 5.0 |
| M | 0.1 | 2.2 | 0.9 | 18.8 | 0.2 | 4.9 | 1.3 | 25.9 |
| N | 0.1 | 1.9 | 0.1 | 1.4 | 0.1 | 2.0 | 0.3 | 5.3 |
| O | 0.2 | 3.2 | 0.3 | 5.9 | 0.1 | 2.1 | 0.6 | 11.2 |
| P | 0.2 | 4.8 | 0.1 | 2.6 | 0.2 | 4.3 | 0.6 | 11.7 |
| Q | 0.1 | 1.1 | 0.0 | 1.0 | 0.2 | 3.1 | 0.3 | 5.2 |
| R | 0.3 | 5.4 | 0.3 | 5.2 | 0.2 | 4.8 | 0.8 | 15.4 |
| S | 0.9 | 17.3 | 2.2 | 44.1 | 1.3 | 26.2 | 4.4 | 87.7 |
| T | 0.5 | 9.6 | 0.8 | 15.3 | 0.5 | 10.6 | 1.8 | 35.4 |
| U | 0.1 | 2.9 | 1.4 | 28.0 | 0.4 | 7.2 | 1.9 | 38.0 |
| V | 0.8 | 16.3 | 0.1 | 3.0 | 0.2 | 4.4 | 1.2 | 23.7 |

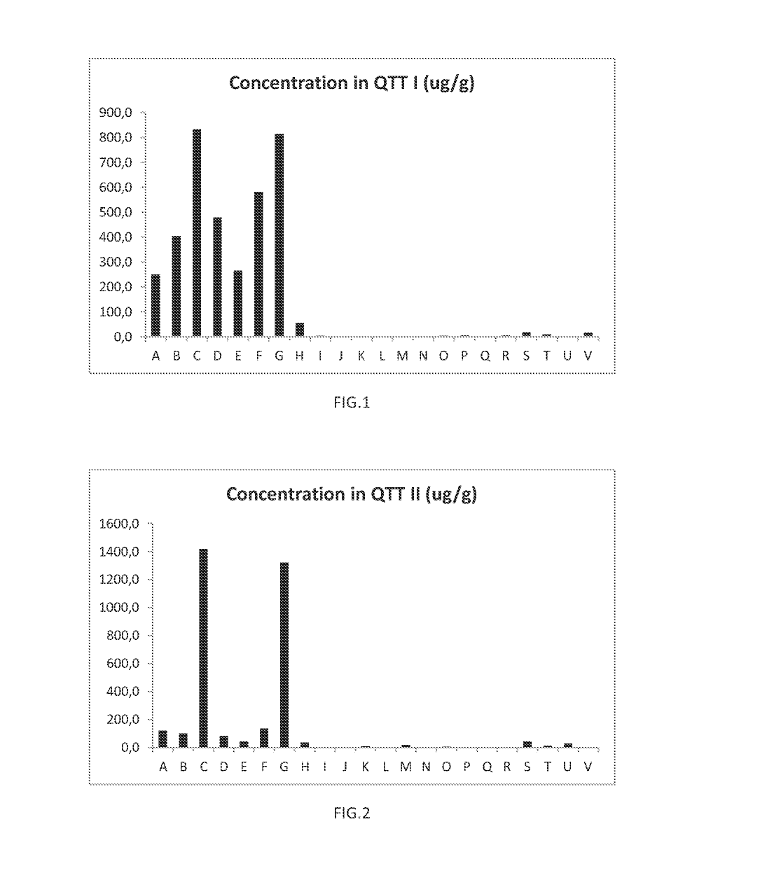

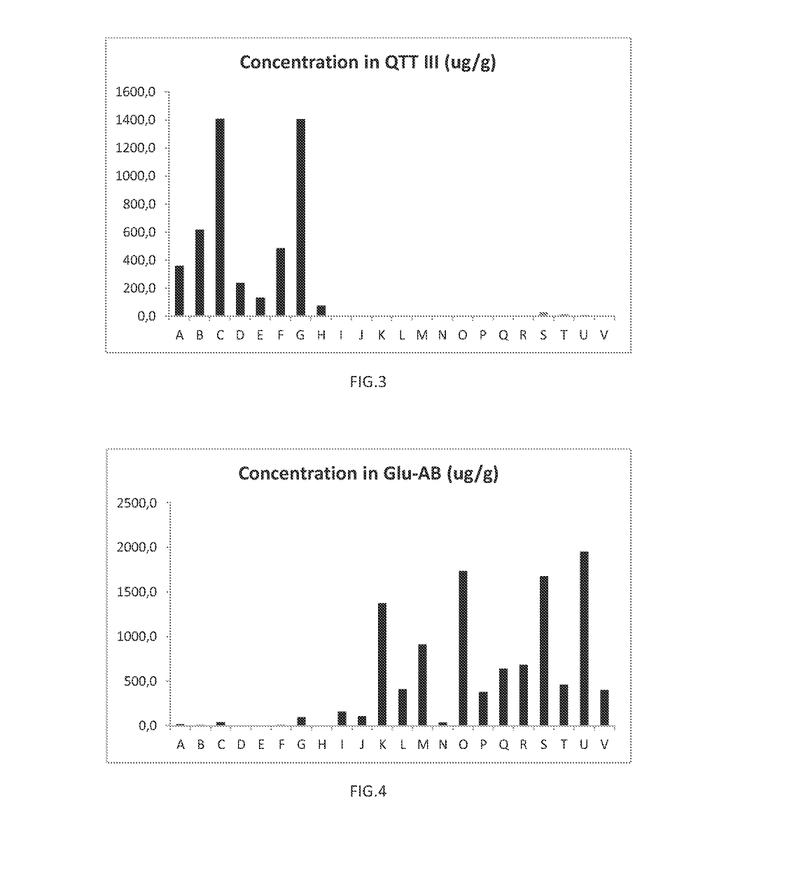

The diagrams of FIGS. 1 to 3 represent respectively the concentrations of QTT I, QTT II and QTT III in the samples A to V.

The diagram of FIG. 5 represents the sum of the concentrations of QTT I, QTT II and QTT III in the samples A to V.

Table 3 below details, for the samples A to V, the concentrations of Glu-AB, said concentrations being expressed in mg/L and in μg/g.

TABLE 3
detailing, for the samples A to V, the concentrations,
in mg/L and in μg/g, of Glu-AB

| | |
|---|---|
| mg/L | μg/g |

Glu-AB

| | | | |
|---|---|---|---|
| Sessile oak | A | 0.9 | 17.6 |
| | B | 0.4 | 8.2 |
| | C | 2.1 | 42.6 |
| | D | 0.3 | 6.8 |
| | E | 0.2 | 4.1 |
| | F | 0.4 | 8.5 |
| | G | 4.9 | 97.9 |
| | H | 0.3 | 6.5 |
| | I | 8.0 | 160.2 |
| Pedunculate oak | J | 5.4 | 108.2 |
| | K | 68.7 | 1373.5 |
| | L | 20.5 | 410.6 |
| | M | 45.5 | 910.5 |
| | N | 1.8 | 36.0 |
| | O | 86.7 | 1733.3 |
| | P | 18.9 | 377.1 |
| | Q | 32.1 | 641.4 |
| | R | 34.1 | 682.8 |
| | S | 83.8 | 1676.2 |
| | T | 23.0 | 460.2 |
| | U | 97.6 | 1952.0 |
| | V | 20.2 | 403.1 |

The diagram of FIG. 4 represents the concentration of Glu-AB in the samples A to V.

Table 4 below details, for the samples A to V, the ratio R and the decimal logarithm of the ratio R, said ratio R corresponding to the sum of the concentrations of QTT I, QTT II, QTT III to the concentration of Glu-AB.

TABLE 4
detailing, for the samples A to V, the ratio
R and the decimal logarithm of the ratio R

| | | R | LOG(R) |
|---|---|---|---|
| Sessile oak | A | 41.40 | 1.62 |
| | B | 137.16 | 2.14 |
| | C | 85.94 | 1.93 |
| | D | 116.36 | 2.07 |
| | E | 108.68 | 2.04 |
| | F | 141.61 | 2.15 |
| | G | 36.16 | 1.56 |
| | H | 25.76 | 1.41 |
| Pedunculate oak | I | 0.07 | −1.14 |
| | J | 0.07 | −1.16 |
| | K | 0.01 | −1.97 |
| | L | 0.01 | −1.91 |
| | M | 0.03 | −1.55 |
| | N | 0.15 | −0.84 |
| | O | 0.01 | −2.19 |
| | P | 0.03 | −1.51 |
| | Q | 0.01 | −2.10 |
| | R | 0.02 | −1.65 |
| | S | 0.05 | −1.28 |
| | T | 0.08 | −1.11 |
| | U | 0.02 | −1.71 |
| | V | 0.06 | −1.23 |

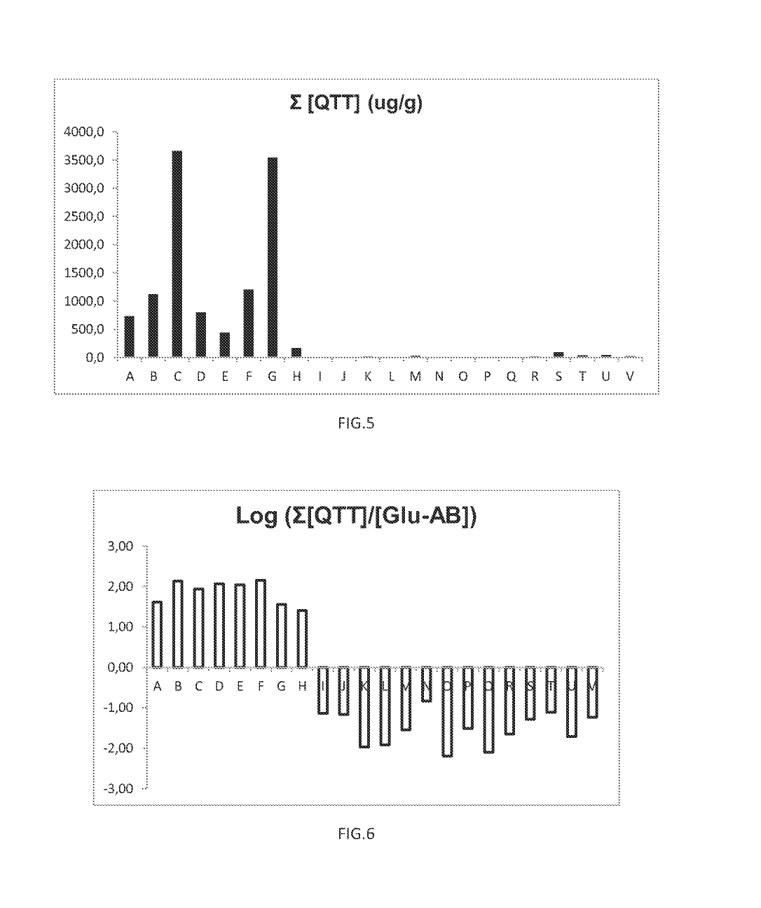

The diagram of FIG. 6 represents the decimal logarithm of the ratio R of the samples A to V.

The values detailed in Table 4 above reveal that all the sessile oak samples (namely the samples A to H) have a value of ratio R higher than 3.162 and all the pedunculate oak samples (namely the samples I to V) have a value of ratio R lower than 0.316.

As regards the decimal logarithm of the ratio R: the decimal logarithm of the ratio R of all sessile oak samples is higher than 0.5 and the decimal logarithm of the ratio R of all pedunculate oak samples is lower than −0.5.

The diagram of FIG. 6 clearly expresses this result of unambiguous differentiation of an oak wood sample of the sessile oak species from the pedunculate oak species.

Moreover, considering Tables 2 and 3 and FIGS. 1 to 5, the samples G and S show all the interest of the method for identifying the oak species of an oak wood sample according to the invention, and in particular the absolute necessity of considering two different categories of molecules which have been described above.

Indeed, the sample G (which actually belongs to the sessile species) has a concentration of Glu-AB which is almost as high as the sample J which belongs to the pedunculate species.

Similarly, the sample S (which actually belongs to the pedunculate species) has a significant concentration of QTT I, QTT II and QTT III which, at first glance, could have led to conclude that it belongs to the sessile species. Indeed, the sessile oak woods have a significant concentration of these molecules of the 1^{st} category of molecules.

Thus, these samples show that, thanks to an appropriate selection of two categories of molecules of derivatives of oleanane-type triterpenes, namely the 1^{st} category of molecules and the 2^{nd} category of molecules which have been detailed above, the identification method according to the invention allows for an unambiguous identification of the oak species of an oak wood.

In other words, these samples show that if only the presence in the wood sample of a molecule belonging either to the 1^{st} category of molecules or to the 2^{nd} category of molecules, is considered, it is possible to erroneously conclude on the oak species of the analyzed sample.

1. A method for identifying the oak species of an oak wood sample, comprises at least the following steps of:
a) Providing an oak wood sample;
b) Preparing the oak wood sample in a suitable manner for an analysis for determining the concentration of derivatives of oleanane-type triterpenes which are present in said oak wood sample;
c) Performing at least one analysis on the thus prepared oak wood sample at step b) so as:
to identify:
i. from one to three molecules belonging to a 1^{st} category of molecules, said one to three molecules being among the three most abundant molecules of all the molecules of said 1^{st} category of molecules present in said oak wood sample, said 1^{st} category of molecules being defined by all derivatives of oleanane-type triterpenes which derive from an aglycone of empirical formula C₃₀H₄₈O₆,
ii. from one to three molecules belonging to a 2^{nd} category of molecules, said one to three molecules being among the three most abundant molecules of all the molecules of said 2^{nd} category of molecules present in said oak wood sample, said 2^{nd} category of molecules being defined by all derivatives of oleanane-type triterpenes which derive from an aglycone of empirical formula C₃₀H₄₆O₇, as well as derivatives of oleanane-type triterpenes which derive from an aglycone of empirical formula C₃₀H₄₆O₈, then,
to determine the concentration:
of each of said one to three molecules of the 1^{st} category of molecules which have been identified;
of each of said one to three molecules of the 2^{nd} category of molecules which have been identified;
d) Determining a ratio R, said ratio R corresponding to the ratio of the concentration or the sum of the concentrations of at least one of said one to three molecules of the 1^{st} category of molecules which have been identified, to the concentration or the sum of the concentrations of at least one of said one to three molecules of the 2^{nd} category of molecules which have been identified;
e) Identifying the species of the oak wood sample according to the value of the ratio R determined at step d) in the following manner:
If the ratio R is lower than 0.316, then the oak wood sample belongs to the pedunculate species,
If the ratio R is higher than 3.162, then the oak wood sample belongs to the sessile species.

2. The identification method according to claim 1, wherein the 1^{st} category of molecules is defined by all derivatives of oleanane-type triterpenes which derive from the arjungenin, the sericic acid, as well as the isomers of the arjungenin and those of the sericic acid.

3. The identification method according to claim 1, wherein the 2^{nd} category of molecules is defined by all derivatives of oleanane-type triterpenes which derive from the bartogenic acid, the isomers of the bartogenic acid, the trachelosperogenin D and the isomers of the trachelosperogenin D.

4. The identification method according to claim 1, wherein the 1^{st} category of molecules is defined by all molecules:
of formula (I), 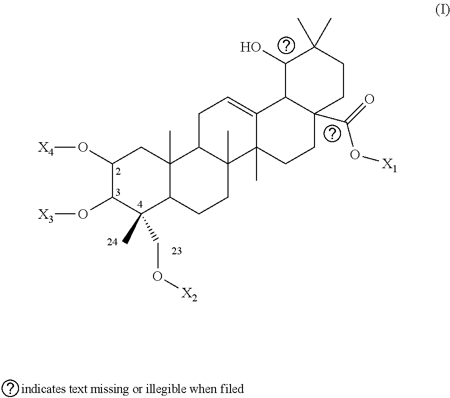
wherein:
X1 represents a glucose group or a hydrogen,
X2 represents a gallate group or a hydrogen,
X3 represents a hydrogen or a glucose group or a gallate group or a glucose-gallate group,
X4 represents a hydrogen or a gallate group,
of formula (II), 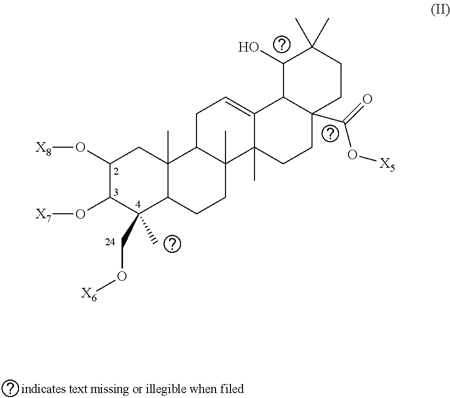
wherein:
X5 represents a glucose group or a hydrogen,
X6 represents a gallate group or a hydrogen,
X7 represents a hydrogen or a glucose group or a gallate group or a glucose-gallate group,
X8 represents a hydrogen or a gallate group.

5. The identification method according to claim 1, wherein the 2^{nd} category of molecules is defined by all molecules:
of formula (III), 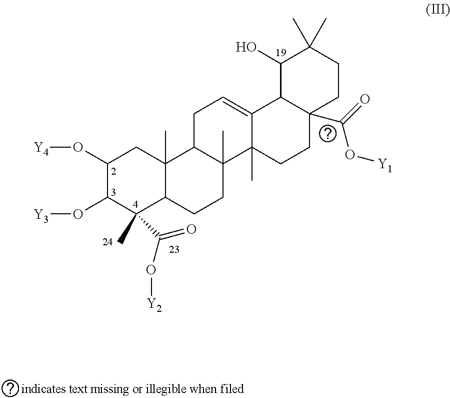
wherein:
Y1 represents a glucose group or a hydrogen,
Y2 represents a glucose group or a hydrogen,
Y3 represents a hydrogen or a gallate group or a glucose group or a glucose-gallate group,
Y4 represents a hydrogen or a gallate group,
of formula (IV), 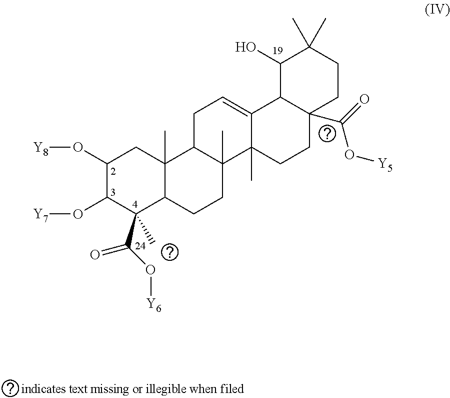
wherein:
Y5 represents a glucose group or a hydrogen,
Y6 represents a glucose group or a hydrogen,
Y7 represents a hydrogen or a glucose group or a gallate group or a glucose-gallate group,
Y8 represents a hydrogen or a gallate group,
of formula (V), 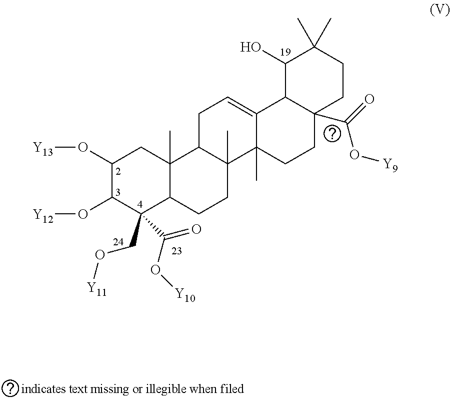
wherein:
Y9 represents a glucose group or a hydrogen,
Y10 represents a glucose group or a hydrogen,
Y11 represents a hydrogen or a gallate group,
Y12 represents a hydrogen or a glucose group or a gallate group or a glucose-gallate group,
Y13 represents a hydrogen or a gallate group,
of formula (VI), 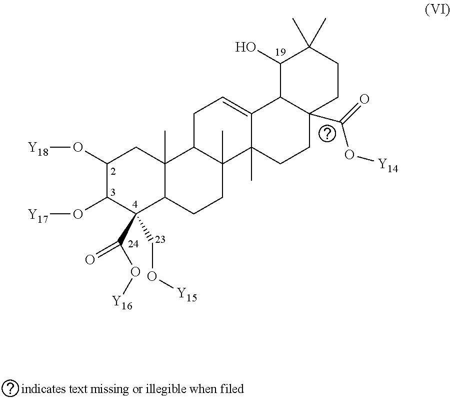
wherein:
Y14 represents a glucose group or a hydrogen,
Y15 represents a hydrogen or a gallate group,
Y16 represents a glucose group or a hydrogen,
Y17 represents a hydrogen or a glucose group or a gallate group or a glucose-gallate group,
Y18 represents a hydrogen or a gallate group.

6. The identification method according to claim 1, wherein the 1^{st} category of molecules is defined by all the following molecules:
the quercotriterpenoside I of formula (VII), 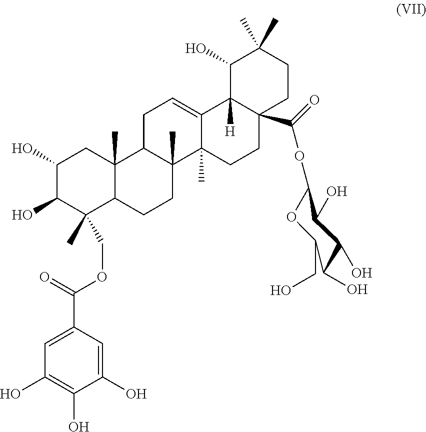
the quercotriterpenoside II of formula (VIII), 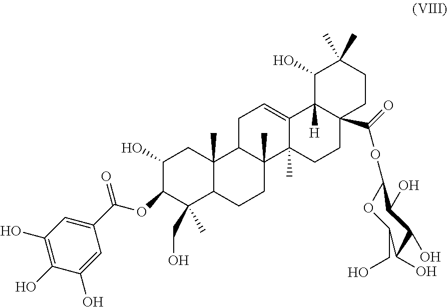
the quercotriterpenoside III of formula (IX),

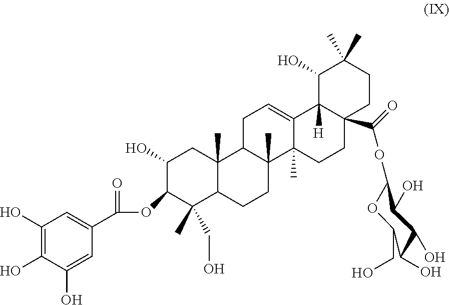

7. The identification method according to claim 1, wherein the 2^{nd} category of molecules is defined only by the 28-glucosylated derivative of the bartogenic acid of formula (X):

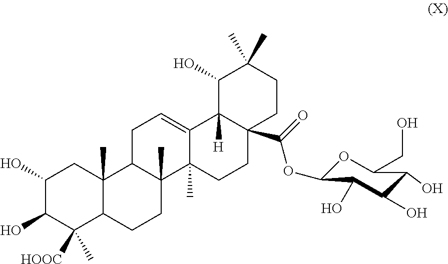

8. The identification method according to claim 7, wherein the ratio R corresponds to the ratio of the concentration or the sum of the concentrations of at least one of the molecules chosen among the quercotriterpenoside I of formula (VII), the quercotriterpenoside II of formula (VIII) and the quercotriterpenoside III of formula (IX), to the concentration of the 28-glucosylated derivative of the bartogenic acid of formula (X).

9. The identification method according to claim 8, wherein the ratio R corresponds to the ratio of the sum of the concentrations of the quercotriterpenoside I of formula (VII), the quercotriterpenoside II of formula (VIII) and the quercotriterpenoside III of formula (IX), to the concentration of the 28-glucosylated derivative of the bartogenic acid of formula (X).

10. The identification method according to claim 1, wherein at step b), the oak wood sample is prepared through a solid-liquid extraction.

11. The identification method according to claim 1, wherein at step c), the analysis consists of at least one analysis chosen among:
liquid chromatography analyses coupled to a mass spectrometry analysis,
gas chromatography analyses coupled to a mass spectrometry analysis,
nuclear magnetic resonance analyses.

12. The identification method according to claim 1, wherein at step e) of the identification method, the decimal logarithm of the ratio R is calculated, and
if the decimal logarithm of the thus calculated ratio R is higher than 0.5 then the oak wood sample belongs to the sessile species;
if the decimal logarithm of the thus calculated ratio R is lower than −0.5 then the oak wood sample belongs to the pedunculate species.
